(19) United States
(12) Patent Application Publication
(10) Pub. No.: US 2007/0134338 A1
(43) Pub. Date: Jun. 14, 2007

(54) Methods for producing nanoparticulate metal complexes and altering nanoparticle morphology

(76) Inventors: Bala Subramaniam, Lawrence, KS (US); Andrew Borovik, Lawrence, KS (US); Chad Johnson, Lawrence, KS (US); Sarika Sharma, Olathe, KS (US)

Correspondence Address: STINSON MORRISON HECKER LLP;ATTN: PATENT GROUP, 1201 WALNUT STREET, SUITE 2800, KANSAS CITY, MO, 64106-2150, US

(21) Appl. No.: 11/449,125

(22) Filed: Jun. 8, 2006

Related U.S. Application Data
(60) Provisional application No. 60/688,478, filed on Jun. 8, 2005.

(51) Int. Cl. A61K 9/14; B29B 9/00

(52) U.S. Cl. 424/489; 264/5; 977/906

(57) ABSTRACT

Nanoparticulate metal complexes, such as those involving ruthenium, iron, cobalt, and nickel salens, are formed using precipitation with compressed antisolvent technology. The nanoparticle morphology may be altered by altering the planarity of molecular structure of the metal complex starting material.

### CROSS-REFERENCE TO RELATED APPLICATIONS

This application is based on and claims priority to U.S. Provisional Application Ser. No. 60/688,478, filed on Jun. 8, 2005, which is hereby incorporated herein by reference.

### STATEMENT REGARDING FEDERALLY SPONSORED RESEARCH OR DEVELOPMENT

The present invention was sponsored by the National Science Foundation Grant No. EEC-0310689, and the government may have certain rights in the invention.

### BACKGROUND OF THE INVENTION

Methods for particle micronization and nanonization by recrystallization, such as those involving precipitation with compressed antisolvent (“PCA”) technology, are set forth in Subramaniam et al., U.S. Pat. No. 5,874,029, which is incorporated by reference. In that patent, nanoparticles comprised of hydrocortisone, RG503H (poly(lactide-co-glycolide)). ibuprofen, camptothecin were prepared. Thus, while various nanomaterials have been prepared using PCA methods, nearly all use organic compounds as the molecular precursors. See Krober H. and Teipel U., Materials processing with supercritical antisolvent precipitation: process parameters and morphology of tartaric acid, J. Supercrit. Fluids, 22, 229-235 (2002); Reverchon E., Supercritical antisolvent precipitation of micro- and nano- particles, J. Supercrit. Fluids, 15, 1-21 (1999); Park Y., Curtis C. W., Roberts C. B., Formation of Nylon Particles and Fibers using Precipitation with a Compressed Antisolvent, Ind. Eng. Chem. Res., 41(6), 1504 (2002). More recently, researchers reported on the preparation of spherical particles ranging between 75 nm and 5 microns in size of an amorphous mixture of an inorganic vanadium phosphate catalyst. Hutchings G. J., Bartley J. K., Webster J. M., Lopez-Sanchez J. A., Gilbert D. J., Kiely C. J., Carley A. F., Howdle S. M., Sajip S., Caldarelli S., Rhodes C., Volta J. C., and Poliakoff M., Amorphous vanadium phosphate catalysts from supercritical antisolvent precipitation, Journal of Catalysis 197, 232-235 ISSN 0021-9517 (2001). To date, the preparation of nanoparticles of metal complex molecules has not been reported.

The present invention involves the preparation of nanoparticles of metal complexes using PCA technology. Moreover, it is surprisingly discovered that the nanoparticle morphology can be dramatically altered by making modifications to the planarity of the molecular structure of the starting material.

### BRIEF SUMMARY OF THE INVENTION

The present invention is directed to nanoparticles comprised of metal complexes and a process of making the nanoparticles. In one aspect, the nanoparticle morphology is altered based on the molecular structure of the precursor compound.

In another aspect, the invention is directed to a process for the production of nanoparticles. The process comprising the step of providing a first compound with a first molecular structure; altering the planarity of the molecular structure of the first compound to form a second compound with a second molecular structure; forming a solution including at least one solvent and at least one solute comprising the second compound with the second molecular structure; spraying the solution containing the at least one solute through a nozzle into an antisolvent; generating atomized droplets of the solution; and contacting droplets with the antisolvent to form nanoparticles of the solute with a particle morphology. In one aspect, the first molecular structure is planar, and the second molecular structure is non-planar so that the nanoparticles become more spherical in shape.

The PCA process is applied to any suitable metal complex starting material. In a preferred embodiment, a metal-salen complex is used as the precursor material. The planarity of the metal salen (e.g. nickel, cobalt, iron, ruthenium salens) may be modified in order to alter the particle morphology of the PCA processed nanoparticles. For example, nickel or cobalt salens having a planar structure may be altered by the addition of an axial group on a ring of the metal salen to form a non-planar metal salen starting material. As another example, the nickel or cobalt salens having a planar structure may be altered by using a different ethylene linker to form a non-planar metal salen starting material.

In another aspect, a process for the production of nanoparticulate metal complexes is provided, and the process comprises: providing a metal complex; forming a solution including at least one solvent and the metal complex; spraying the solution containing the at least one solvent and metal complex through a nozzle into an antisolvent; generating atomized droplets of the solution; and contacting droplets with the antisolvent to form a nanoparticulate metal complex.

Exemplary metal complexes include transition metals complexed with a salen, saltin, salophen, or salayhexin ligand. The nanoparticulate metal complex may have an elongated rod structure or a generally spherical structure.

In still another aspect, a process for altering the morphology of a nanoparticle is provided. The process includes providing a first compound with a first molecular structure and forming a nanoparticle of the first compound having a first particle morphology using PCA technology. The planarity of the first compound is altered to form a second compound having a second molecular structure. PCA technology is then used to form a nanoparticle having a second particle morphology that is different than the first particle morphology. For example, the first molecular structure may planar, which results in a rod-like nanoparticle morphology. The molecular structure is altered to from a non-planar molecule, which results in a second particle morphology comprising spheres.

Additional aspects of the invention, together with the advantages and novel features appurtenant thereto, will be set forth in part in the description that follows, and in part will become apparent to those skilled in the art upon examination of the following, or may be learned from the practice of the invention. The objects and advantages of the invention may be realized and attained by means of the instrumentalities and combinations particularly pointed out in the appended claims.

### BRIEF DESCRIPTION OF THE DRAWINGS

FIG. 1 shows the x-band electron paramagnetic resonance (“EPR”) spectra measured at 77 K for solid samples of processed Co(II)(salen) (dashed line) and unprocessed Co(II)(salen) (solid line).

FIG. 2 shows the electronic absorption spectra of processed Co(II)(salen) (dashed line) and unprocessed Co(II)(salen) (solid line) suspended in phosphate buffer solution (0.05 M, pH of 7.2).

FIG. 3 shows the electronic absorption spectra of processed Ni(II)(salen) (dashed line) and unprocessed Ni(II)(salen) (solid line) suspended in phosphate buffer solution (0.05 M, pH of 7.2).

FIG. 4 shows the x-band EPR spectra measured at 77 K for solid samples of processed Ru(salen)(NO)(Cl) (solid line) and unprocessed Ru(salen)(NO)(Cl) (dashed line).

FIG. 5 shows the electronic absorption spectra of processed Ru(salen)(NO)(Cl) (dashed line) and unprocessed Ru(salen)(NO)(Cl) (solid line) suspended in phosphate buffer solution (0.05 M, pH of 7.2).

FIG. 6 is an SEM of unprocessed Ni(II)salen (right panel) and the processed rod-like nanoparticles of Ni(II)salen (left panel).

FIG. 7 is a SEM of the processed Co(II)salen. The nanoparticles have an elongated rod-like structure.

FIG. 8 is a SEM of the processed Ni(II)(salen*) irregular elongated nanoparticles derived from the nonplanar Ni(II)(salen*) starting material.

FIG. 9 is a SEM of the unprocessed Ru(salen)(NO)(Cl) irregular particles comprised of amorphous shards (left panel) and processed Ru(salen)(NO)(Cl) spherical nanoparticles (right panel).

### DETAILED DESCRIPTION OF PREFERRED EMBODIMENT

The present invention is directed to nanoparticulates comprised of metal complexes. The nanoparticulate metal complexes are preferably prepared using precipitation with compressed antisolvent (“PCA”) technology. The PCA technique is a semi-continuous method that regularly utilizes a supercritical fluid, such as supercritical carbon dioxide, as the precipitant. Exemplary PCA techniques are set forth in Subramaniam et al., U.S. Pat. No. 5,874,029, which is incorporated by reference. See also Subramaniam B., Rajewski R. A., and Snavely W. K., Pharmaceutical processing with supercritical carbon dioxide, J. Pharm. Sci., 86, 885-890 (1997); Perrut, Supercritical fluids applications in the pharmaceutical industry, STP Pharma Sciences, 13, 83-91 (2003); Foster N. R., Mammucari R., Dehghani F., Barrett A., Bezanehtak K., Coen E., Combes G., Meure L., Ng A., Regtop H., and Tandya A., Processing pharmaceutical compounds using dense-gas technology, J. Ind. Eng. Chem. Res., 42 (25), 6476-6493 (2003); Rehman M., Shekunov B. Y., York P., Lechuga-Ballesteros D., Miller D. P., Tan T., and Colthorpe P., Optimisation of powders for pulmonary delivery using supercritical fluid technology, Eur. J. Pharm. Sci., 22(1), 1-17 (May 2004). In general, during processing, carbon dioxide dissolves into a solution of the desired compound as the solvent diffuses out. See Lin C., Muhrer G., Mazzotti M., and Subramaniam B., Vapor-liquid mass transfer during gas antisolvent recrystallization: Modeling and experiments, Ind. Eng. Chem. Res., 42 2171 (2003). Because of the greater precipitant-solvent ratio and efficient mass transfer, substantial supersaturation is achieved, resulting in the production of small, relatively uniform particles of the dissolved compound.

As used herein, the term “supercritical fluid” means either a fluid simultaneously above its critical temperature (T_{c}) and pressure (P_{c}), or a fluid suitable for use as a supercritical antisolvent. In the practice of the present invention, and as used herein, “supercritical fluid,” means the temperature of the fluid is in the range of 1.01 T_{c} to 5.0 T_{c} and the pressure of the fluid is in the range of 1.01 P_{c} to 8.0 P_{c}. In a most preferred embodiment, the temperature of the fluid is in the range of 1.01 T_{c} to 1.2 T_{c} and the pressure of the fluid is in the range of 1.01 P_{c} to 2.0 P_{c}.

As used herein, the term, “nanoparticle” or “nanoparticulate” means a particle having at least one dimension that is less than about 1 micron. An example of a “nanoparticle” is a generally spherical particle with a diameter less than 1 micron. Another example of a “nanoparticle” is a rod-like elongated structure having a diameter of 1-10 nm, but a length greater than 1 micron because at least one dimension is less than 1 micron.

As used herein, the term “planar” means that the geometry of is generally confined to two dimensions on a single plane.

As used herein, the term “metal complex” means a discrete molecule that contains a metal ion and a ligand. In one aspect, the metal complexes are coordination compounds. In another aspect, the metal complexes are “organometallic complexes,” meaning that the complex is between the metal ion and a carbon on a ligand comprising a carbon-containing compound.

In one aspect, the nanoparticulate metal complexes of the present invention are generally spherical in shape and have an average diameter less than about 500 nm, 300 nm, 200 nm, 100 nm, 80 nm, 50 nm, 40 nm, 30 nm, or 10 nm.

In another aspect, the nanoparticulate metal complexes of the present invention are elongated rod-like structures. In one aspect, the average length of the rod is greater than 1 micron, but the average diameter is on the order of about 200 nm. In still another aspect, the elongated rod-like structure has a submicron length and an average diameter less than about 100 nm. In yet another aspect, the elongated rod-like structure has an average length of about 700 nm, and an average diameter of about 85 nm.

In the present invention, the PCA methodology was used to prepare nanoparticulate metal complexes from a metal complex starting material. Suitable metals for forming the metal complex starting materials of the present invention include the transition metals, e.g. Co, Cr, Fe, V, Mg, Ni, Ru, Zn, Al, Sc, Zr, Ti, Sn, La, Os, Yb, and Ce. Preferred transition metal ions are selected from the group consisting of manganese, nickel, cobalt, iron, and ruthenium.

In one aspect, the metal is complexed to a bidentate, tridentate, or tetradentate ligand. In a preferred aspect, the metal is complexed to a tetradentate ligand.

Exemplary ligands include organic molecules, such as salens, metalloporphyrin, phthalocyanine, macrocyclic teraaza, and cyclam-type ligand systems as set forth in Cuellar et al., U.S. Pat. No. 4,668,349, which is incorporated by reference. Most preferably, the ligand is a “salen.” The term “salen” is a contraction used to refer to those ligands typically formed through a salicylic aldehyde derivative with one molecule of a diamine derivative. While salen ligands are formed from ethylenediamine derivatives, propyl and butyl diamines may also be used to give analogous salpn and salbn derivatives. Exemplary ligands for complexing the metals are set forth in U.S. Pat. Nos. 5,665,890, 5,929,232, 5,663,393 and 5,637,739, all to Jacobsen et al., which are incorporated by reference, and Lui et al., U.S. Pat. No. 6,693,206, which is incorporated by reference.

In the most preferred embodiment of the invention, the metal complex comprises a transition metal anion (preferably Mn, Ni, Ru, Co) and an organic ligand selected from N,N′-bis(salicylaldehyde/substituted salicylaldehyde) ethylenediimine (salen), N,N′-bis(salicylaldehyde/substituted salicylaldehyde) 1,3-propylenediimine (saltin), N,N′-bis(salicylaldehyde/substituted salicylaldehyde) 1,2-phenylenediimine (salophen or salph), N;N′-bis(salicylaldehyde/substituted salicylaldehyde) 1,2-cyclohexane diimine (salcyhexen), and their unsubstituted or substituted derivatives. Suitable methods of substituting and altering the molecular structure of these ligands and the corresponding metal complexes is set forth in U.S. Pat. Nos. 5,665,890, 5,929,232, 5,663,393 and 5,637,739, all to Jacobsen et al., which are incorporated by reference, and Lui et al., U.S. Pat. No. 6,693,206, which is incorporated by reference.

Most preferred metal complexes are those selected from the group consisting of [N,N′-ethylenebis(salicylidene-aminato(2-)]cobalt(II) (hereinafter referred to as “Co(II)(salen)”); [N,N′-ethylenebis(salicylidene-aminato(2-)]nickel(II) (hereinafter referred to as “Ni(II)(salen)”); and [N,N′-Bis(3,5-di-tert-butylsalicylidene)1,2-cyclohexanediaminato(2-)]nickel(II) (hereinafter referred to as “Ni(salen*)”).

Salens and their derivatives have various functions, ranging from reversible gas binders. See Jones R. D., Summerville R. A., and Basolo F. (1979), Chem. Rev., 79, 139-179; Niederhoffer E. C., Timmons J. H., Martell A., Thermodynamics of oxygen binding in natural and synthetic dioxygen. complexes, Chem. Rev., 84, 137-203 (1984); Norman A.T., Pez G. P., and Roberts D. A., in Martell A. E., and Sawyer D. T. (Eds.), Oxygen Complexes and Oxygen Activation by Transition Metals, 107-127 (1988). In addition, salens and their derivatives are also used as enantioselective catalysts. See Jacobsen E. N., Asymmetric Catalysis of Epoxide Ring-Opening Reactions, Acc. Chem. Res., 33, 421-431 (2000).

As discussed more fully below, the metal complex starting material is subject to PCA processing to form a nanoparticulate metal complex. In one aspect, the molecular geometry of the metal complex starting material is altered in order to alter the morphology of the processed nanoparticle. For example, it has been found that a transition metal complex having a planar structure, such as Ni(II)salen and Co(II)salen, subjected to PCA processing will form an elongated rod-like nanoparticle. When modifications to the molecular geometry of the precursor material are made so that the metal complex is no longer planar, PCA processing results in deviations from the rod geometry. In particular, modifications to the ethylene linker and/or additions to the aromatic rings (e.g. as in the case of the Ni(II)(salen*)) result in the formation of elongated irregular shaped nanoparticles. Similarly, axial substituents on the metal complex (e.g. as in the case of Ru(salen(NO)(Cl)) result in the formation of spherical nanoparticles.

The following examples are provided by way of explanation and illustration. As such, these examples are not to be viewed as limiting the scope of the invention.

### EXAMPLE 1

A. Salen Complex Preparation

All reagents were purchased from commercial sources and used as received, unless otherwise noted. Syntheses of some complexes were conducted in a Vacuum Atmospheres Dry box under an argon atmosphere. Standard Schlenk techniques were used during the work-up of some reactions and manipulations of samples outside the dry box. NMR spectra were recorded on Bruker DRX400 400 MHz spectrometers equipped with Silicon Graphics workstations. Electronic absorbance spectra were recorded with a Cary 50 spectrophotometer using a 1.00 cm quartz cuvet. FTIR spectra were collected on a Mattson Genesis series FTIR instrument with values reported in wavenumbers. EPR spectra were collected using a Bruker EMX spectrometer equipped with an ER4102ST cavity.

Co(II)(salen) was purchased from Aldrich (23,606-3). EPR: (X-band, solid, 77 K) g =2.00. λₘₐₓ/nm: (phosphate buffer solution (aq), suspension) 250, 376. ELEMENTAL ANALYSIS: Theoretical (%) C, 59.09, H, 4.34, N, 8.61, Co, 18.12. Experimental (%) C, 57.08, H, 4.25, N, 8.25, Co, 17.02.

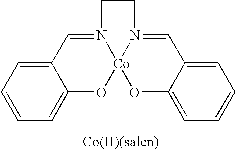

Ni(II)(salen) was prepared as follows. To a 500 mL round bottom flask was added 2.2196 g (8.2822 mmol) of salen (N,N′-disalicylideneethylenediamine) that was partially dissolved in 200 mL of a 1:1 solution of THF and water to give a yellow suspension. To this mixture was added 2 equivalents of K₂CO₃ (2.2297 g, 16.133 mmol) and 1 equivalent of Ni^{II}(OAc)₂4H₂O (2.0516 g, 8.2440 mmol) simultaneously. The reaction was stirred for 18 hours at room temperature and pressure as the solution color changed from yellow to dark orange. The solid product was filtered using a 60 mL fine frit and washed with diethyl ether and then water until the filtrate became clear. The solid was dried under vacuum overnight. The yield of the complex was 2.4108 g (90%). ¹H NMR: (CDCl₃, ppm) δ=3.44 (s, 4H, CH₂CH₂); 6.52-6.56 (m, 2H, salicyl phenyl); 7.03-7.05 (d, 2H, salicyl phenyl); 7.06-7.09 (dd, 2H, salicyl phenyl); 7.19-7.62 (m, 2H, salicyl phenyl); 7.51 (s, 2H, —NCH(Ph)). λₘₐₓnm: (DMSO) (ε, M⁻¹ cm⁻¹) 390 (4233), 408 (6573), 440 (3390), 540 (121), see also Freire C. and Castro B., Spectroscopic characterisation of electrogenerated nickel(III) species. Complexes with N₂O₂ Schiff-base ligands derived from salicylaldehyde., J. Chem. Soc., Dalton Trans., 1491-1498 (1998); (suspension in phosphate buffer solution) 250, 323, 389. IR: (KBr, cm⁻¹) 469 (Ni—N), 411 (Ni—O), see Garg B. S. and Nandan Kumar D., Spectral studies of complexes of nickel(II) with tetradentate schiff bases having N₂O₂ donor groups, Spectrochimica Acta Part A, 59, No. 2, 22.9-234(6) (15 Jan. 2003). ELEMENTAL ANALYSIS: Theoretical (%) C 59.13, H 4.34, N 8.62, Ni 18.06. Experimental (%) C 58.93, H 4.35, N 8.44, Ni 17.12.

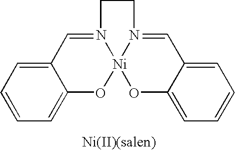

Ru(NO)Cl₃ was synthesized following a similar procedure described by Mitchell-Koch J. T., Reed T. M., and Borovik A. S., Light-Activated Transfer of Nitric Oxide From a Porous Material, Angew. Chem. Int. Ed., 43(21), 2806-2809 (2004). See also Muller, J. G.; Takeuchi, J. K., Preparation and Characterization of Trans-bis(alpha-dioximato)Ruthenium Complexes, Inorg. Chem. 29, 2185-2188 (1990). RuCl₃xH₂O (3.028 g) was dissolved in 75 mL of 1 M HCl and the solution was degassed with nitrogen for 10 minutes. The mixture was brought to reflux and an aqueous solution (35 mL) of NaNO₂ (3.0171 g, 43.726 mmol) was added dropwise. After 4 hours of reflux, the solution was cooled to room temperature and the solvent was removed under reduced pressure. The red/brown residue was dissolved in 35 mL of ethanol and filtered to remove excess salts. The filtrate was washed using 6 M HCl and then 25 mL of water with the solvent being removed after each wash under reduced pressure. This final water washing was repeated three times. The final salt was dried in a vacuum oven at 60° C. to yield 3.275 g (95%) of a red brown solid. IR: (Nujol, cm⁻¹) 1898 (NO).

Ru(salen)(NO)(Cl) This complex was synthesized following a similar procedure described by Mitchell-Koch J. T., Reed T. M., and Borovik A. S., Light-Activated Transfer of Nitric Oxide From a Porous Material, Angew. Chem. Int. Ed., 43(21), 2806-2809 (2004). See also Works, C. F.; Ford, P. C. Photoreactivity of the ruthenium nitrosyl complex, Ru(salen)(Cl)(NO), Solvent effects on the back reaction of NO with the Lewis acid Ru^{III}(salen)(Cl), J. Am. Chem. Soc., 122, 7592-7593 (2002); Works, C. F., Jocher, C. J., Bart, G. D., Bu, X. & Ford, P. C. Photochemical nitric oxide precursors: synthesis, photochemistry, and ligand substitution kinetics of ruthenium salen nitrosyl and ruthenium salophen nitrosyl complexes. Inorg. Chem., 41, 3728-3739 (2002); Bordini, J., Hughes, D. L., Da Motta Neto, J. D. & da Cunha, C. J. Nitric oxide photorelease from ruthenium salen complexes in aqueous and organic solutions, Inorg. Chem., 41, 5410-5416 (2002). Under an argon atmosphere, a 50 mL DMF solution of salen (1.0034 g, 3.7441 mmol) was treated with 2 equivalents of solid KH (0.300 g, 7.48 mmol). After H₂ evolution was completed (about 30 minutes), Ru(NO)Cl₃ (0.890 g, 3.75 mmol) was added. This reaction mixture was taken out of the dry box and was refluxed for 2 hours under N₂. The DMF was removed under reduced pressure and the solid residue was allowed to cool overnight. The brown solid was further purified using silica gel flash chromatography with a mobile phase of 2% methanol/98% CH₂Cl₂. Fractions containing Ru(salen)(NO)(Cl) were combined and the solvent was removed under reduced pressure to yield 0.724 g (60%) of a brown solid. ¹H NMR: (CDCl₃, ppm) δ=3.97-4.02 and 4.36-4.41 (dd, 4H, CH₂CH₂); 6.68-6.71 (t, 2H, salicyl phenyl); 7.24-7.26 (d, 2H, salicyl phenyl); 7.30-7.32 (d, 2H, salicyl phenyl); 7.41-7.45 (t, 2H, salicyl phenyl); 8.26 (s, 2H, —NCH(Ph)). IR: (KBr, cm−1) 1832 (NO), 1603 (C═N), 1520 (C═C), see Works C. F., Jocher C. J., Bart G. D., Bu X., and Ford P. C., Photochemical Nitric Oxide Precursors: Synthesis, Photochemistry, and Ligand Substitution Kinetics of Ruthenium Salen Nitrosyl and Ruthenium Salophen Nitrosyl Complexes, Inorg. Chem., 41(14), 3728-3739 (2002). λₘₐₓ/nm: (CH₂Cl₂) 378; (suspension in phosphate buffer solution, nm) 249, 271 (sh), 383.

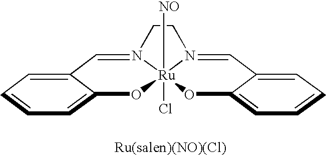

(R,R)-N,N′-Bis(5-3-tert-butyl-salicylidene)-1,2-cyclohexanediamine was synthesized following a similar procedure described by Jacobsen E. N., Zhang W., Muci A. R., Ecker J. R., and Deng L. Highly Enantioselective Epoxidation Catalysts Derived from 1,2-Diaminocyclohexane, J. Am. Chem. Soc., 113, 7063-7064 (1991). To a 250 mL round bottom flask was added 2.0053 g (8.5573 mmol) of 3,5 di-tert butyl-2-hydroxybenzaldehyde that was dissolved in 20 mL absolute ethanol. Concurrently, (R,R)-1,2-diammoniumcyclohexane mono-(+)-tartrate salt (see Larrow J. F., Jacobsen E. N., Gao Y., Hong Y., Nie X., and Zepp C. M., A Practical Process for the Large-Scale Preparation of (R,R)-N,N′-Bis(3,5-Di-tert-butylsalicylidene)-1,2-Cyclohexanediaminomanganese (III) Chloride, a Highly Enantioselective Epoxidation Catalyst, J. Org. Chem., 59, 1939-1940 (1994); (1.1219 g, 4.2451 mmol) was dissolved in a basic (NaOH) 0.2 M aqueous/absolute ethanol solution (1:2). This salt solution was added dropwise to the benzaldehyde solution and the mixture was refluxed under nitrogen for 1 hour. The reaction mixture was filtered using a 60 mL medium frit and washed with 95% ethanol. The product was then extracted into methylene chloride. The frit was washed with additional methylene chloride until the solid was colorless. The solvent was removed under reduced pressure to yield 1.6843 gm (70%) of a yellow solid. ¹H NMR: (CDCl₃, ppm) δ=1.24 (s, 9H); 1.41 (s, 9H); 1.45 (m, 1H); 1.65-1.8 (m, 1H); 1.8-2.0 (m, 2H); 3.32 (m, 1H); 6.98 (d, 1H); 7.30 (d, 1H); 8.30 (s, 1H); 13.72 (s, 1H).

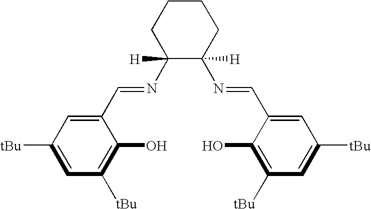

(R,R)-N,N′-Bis(5-3-tert-butyl-salicylidene)-1,2-cyclohexanediamine

Ni(salen*) was prepared as follows: First, to a 250 mL round bottom flask was added 1.0087 g (1.8446 mmol) of (R,R)-N,N′-Bis(5-3-tert-butyl-salicylidene)-1,2-cyclohexanediamine that was dissolved in 44 mL of methylene chloride. Concurrently, Ni^{II}(OAc)₂4H₂O (0.5076 g, 2.040 mmol) was dissolved in 25 mL of dry methanol. The nickel solution was added dropwise to the reaction mixture was stirred for 2 hours at room temperature. The mixture was then cooled to 3° C. in an ice bath and stirred for an additional 0.5 hour. The solid product was filtered using a 60 mL medium frit and washed with cold dry methanol until the filtrate became clear. The solid was dried under vacuum overnight. The yield of the complex was 0.7316 g (66%). ¹H NMR: (CDCl₃, ppm) μ=1.28 (s, 9H); 1.34 (m, 2H); 1.43 (s, 9H); 1.92 (m, 1H); 2.45 (m, 1H); 2.99 (m, 1H); 6.90 (d, J=2.4Hz, 1H); 7.31 (d, 1H); 7.40 (s, 1H). ELEMENTAL ANALYSIS: Theoretical (%) C, 71.41, H, 8.99, N, 4.63, Ni, 9.69. Experimental (%) C, 71.72, H, 8.66, N, 4.57, Ni, 9.55.

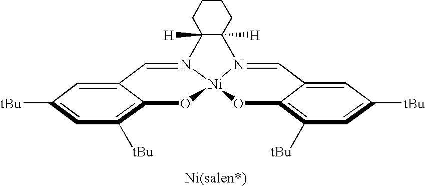

B. PCA Processing

The details of the PCA apparatus used to prepare the nanoparticles have been described previously. See generally Snavely W. K., Subramaniam B., Rajewski R. A., and Defelippis M. R., Micronization of insulin from halogenated alcohol solution using supercritical carbon dioxide as an antisolvent, J. Pharm. Sci., 91, 2026-2039 (2002); Fusaro F., Hänchen M., Mazzotti M., Muhrer G., and Subramaniam B., Dense Gas Antisolvent Precipitation: A Comparative Investigation of the GAS and PCA Techniques, Industrial and Engineering Chemistry Research, 44, 1502-1509 (2005). In general, the procedure involved carbon dioxide, flowing in parallel from two dip tube cylinders, and compressed to the operating pressure by a pneumatically operated gas booster. After passing through a surge tank immersed in a temperature controlled water bath, where pressure fluctuations are dampened, it enters a narrow 2.5 L precipitation vessel also in the same water bath as the surge tank through the converging-diverging annulus of a co-axial nozzle. The solvent methylene chloride (CH₂Cl₂) containing the dissolved metal complex, is supplied at a constant flow rate by a syringe pump (Isco 314) and fed through the inner capillary of the nozzle (152.4 microns). The co-axial carbon dioxide stream in the converging-diverging nozzle rapidly disperses the liquid jet and precipitation takes places at the exit of the nozzle. A stainless steel insert was fabricated to decrease dead volume in the precipitation chamber and direct the flow towards the outlet. The particles are collected outside of the precipitation vessel on a filter unit (0.2 microns), also maintained at constant temperature by being immersed in the same water bath as surge tank and precipitation vessel. Particles with dimensions smaller than 0.2 microns, such as Ru(salen)(CO)(Cl) can also be captured by the filter due to particle agglomeration as is evident through visual inspection of the SEM images. The carbon dioxide-solvent mixture is depressurized across a heated backpressure regulator and the solvent recovered in a glass cyclone. Following the cessation of spraying the organic solvent, additional carbon dioxide was sent through the system to ensure the removal of residual solvent from the processed particles. The system was then depressurized to atmospheric pressure and the particles were harvested. All processing runs were conducted above the pseudo-binary critical locus. See Reverchon E., Caputo G., De Marco, and Revista I., Role of phase behavior and atomization in the supercritical antisolvent, Precipitation, Industrial and Engineering Chemistry Research, 42 (25), 6406-6414 (2003). The variation on processing conditions are shown in the table below:

TABLE S1
Typical Processing Conditions

| | Chamber | Chamber | Solution | Solution | CO2 |
|---|---|---|---|---|---|
| | Pressure | Temperature | Concentration | Flow Rate | Flow Rate |
| System | (±0.3 bar) | (±0.5° C.) | (mg/mL) | (g/min) | (g/min) |
| Ni(II)(salen) | 85 | 40 | 10 | 1.93 | 108.4 |
| Co(II)(salen) | 80 | 37 | 5.3 | 1.93 | 108.4 |
| Ru(salen)(NO)(Cl) | 81 | 38 | 5.1 | 1.93 | 108.4 |
| Ni(II)(salen*) | 81 | 37 | 9.9 | 1.93 | 108.4 |

As discussed below, the results from analytical and spectroscopic studies indicate that the metal complexes remain intact after processed into particles. Further, elemental percentages of the particles obtained from combustion and inductively coupled plasma analyses are in agreement with ratio calculated for their corresponding parent complexes. The data for the PCA processed material is shown below.

PCA processed Co(II)(salen). EPR: (X-band, solid, 77 K) g=2.00. λₘₐₓ/nm: (suspension in phosphate buffer solution) 250, 374. ELEMENTAL ANALYSIS: Theoretical (%) C, 59.09, H, 4.34, N, 8.61, Co, 18.12. Experimental (%) C, 56.92, H, 4.13, N, 8.11, Co, 16.66.

PCA processed Ni(II)(salen). λmax/nm: (suspension in phosphate buffer solution) 250, 323, 389. ELEMENTAL ANALYSIS: Theoretical (%) C, 59.13, H, 4.34, N, 8.62, Ni, 18.06. Experimental (%) C, 58.76, H, 4.25, N, 8.42, Ni, 17.65.

PCA processed Ru(salen)(NO)(Cl). This material is diamagnetic so the EPR signal should be silent as shown in Figure S6. λₘₐₓ/nm: (suspension in phosphate buffer solution) 240, 265 (sh), 361. IR: (KBr, cm⁻¹) 1833 (NO), 1602 (C═N), 1529 (C═C).

PCA processed Ni(II)(salen*). ELEMENTAL ANALYSIS: Theoretical (%) C, 71.41, H, 8.99, N, 4.63, Ni, 9.69. Experimental (%) C, 71.73, H, 8.70, N, 4.62, Ni, 9.05.

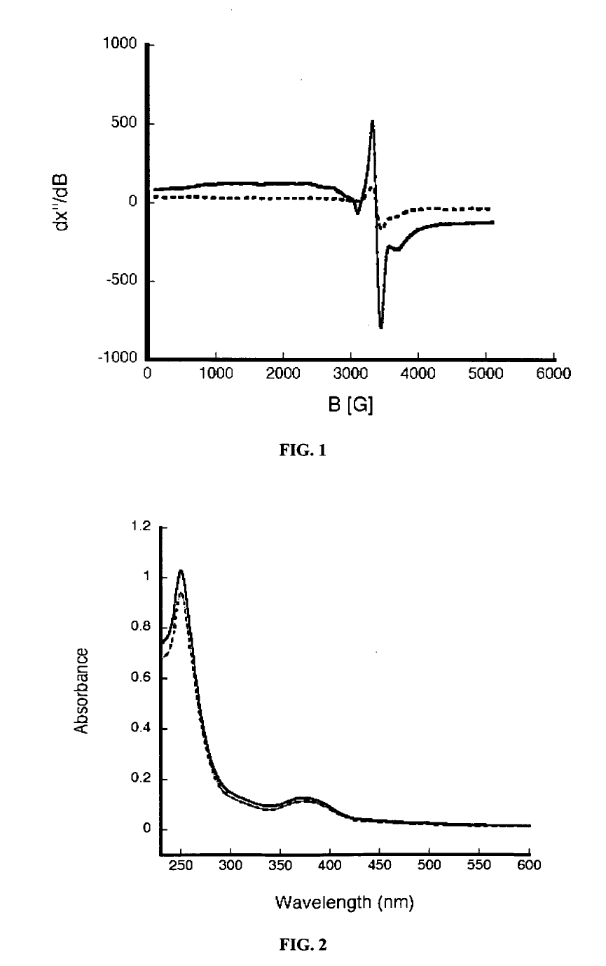

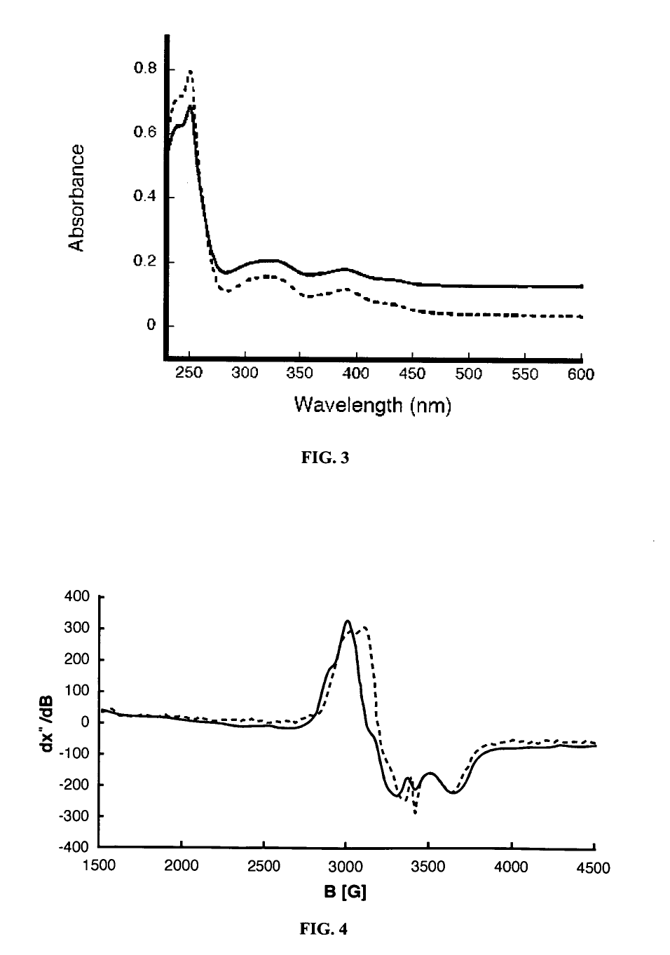

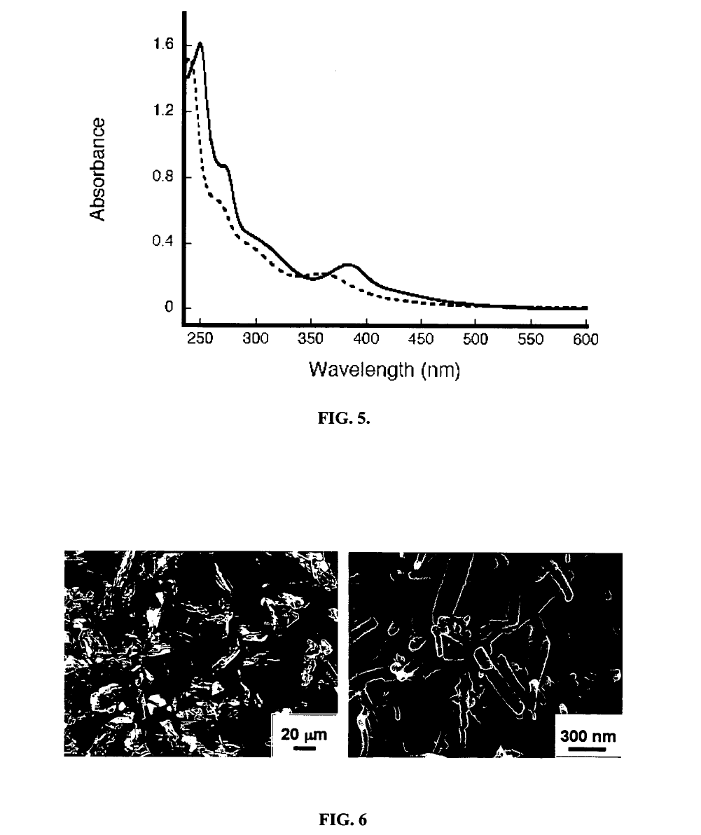

As shown in FIGS. 1 to 5, electronic absorbance and electron paramagnetic resonance measurements of the processed particles are similar to those found for their molecular precursors. For instance, as shown in FIG. 1, particles of processed Co(II)(salen) have an axial X-band electron paramagnetic resonance spectrum (EPR) spectrum that is similar to that of the unprocessed complex.

Scanning electron microscopy (SEM) was used to characterize the structures of the unprocessed and processed particles. As shown in FIG. 6 (right panel), the SEM image of the unprocessed Ni(II)(salen) depicted flat irregular shards with sizes ranging from microns to millimeters. Magnification of these shards did not reveal the presence of discrete primary particles; rather, only amorphous surfaces were observed. A representative SEM image of the processed Ni(II)(salen) complex (FIG. 6, left panel) shows aggregates of primary particles having rod-like structures, with average diameter and length of 85 nm and 700 nm, respectively.

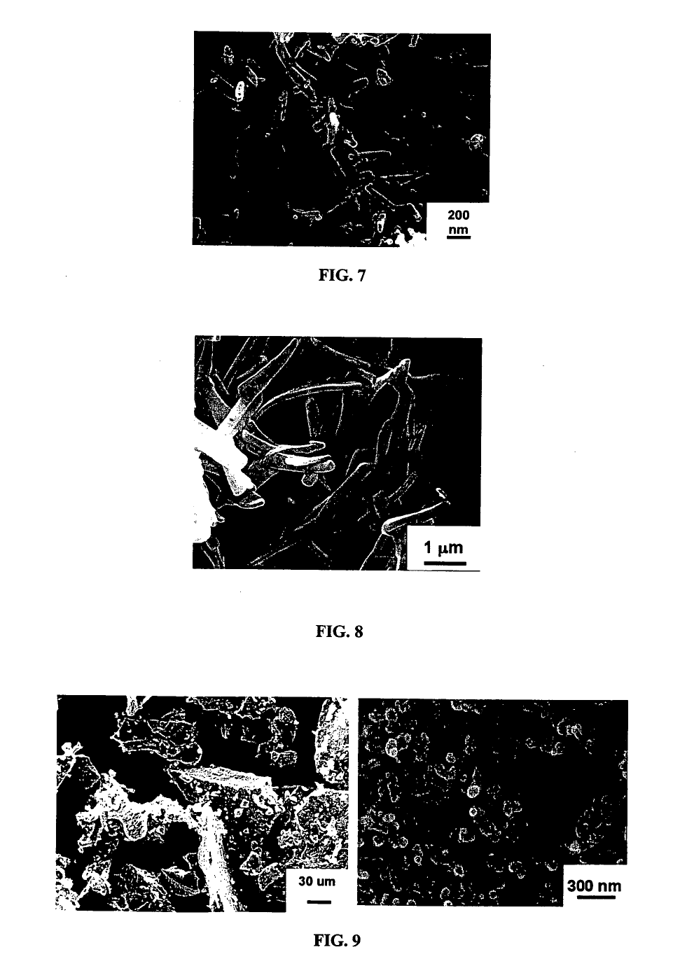

The results were similar for both the planar Ni(II)(salen) and Co(II)(salen) metal complexes. As shown in FIG. 7, PCA processed particles of Co(II)(salen) were also rods with dimensions nearly identical to those observed for the Ni(II)(salen) nanoparticles.

The morphology of the processed nanoparticles changed dramatically when the planarity of the starting material was altered. Ni(II)(salen*) has both tertbutyl substitutions on the aromatic rings and changes to the ethylene linker. As shown in FIG. 8, the primary particles produced from Ni(II)(salen*), the complex having a non-planar, optically-pure salen ligand, were no longer rod-like structures. Instead, the primary particles of Ni(II)(salen*) had irregular shapes that were elongated with micron sized lengths and average diameters on the order of 200 nm.

The core salen structure was also altered with Ru(salen)(NO)(Cl) by providing additional substituents in the axial positions, affording a non-planar molecular structure. As with the planar salen complexes, the unprocessed Ru(salen)(NO)(Cl) gave amorphous shards of varied sizes and shapes (FIG. 9, left panel). After undergoing PCA processing, the resulting primary nanoparticles of Ru(salen)(NO)(Cl) had spherical morphology with an average particle diameter of 50 nm (FIG. 9, right panel).

The foregoing shows that particle morphology may be altered by changing the initial molecular geometry of the compound being precipitated. These results suggest that there is a correlation between the molecular structure of the precursor and the final morphology of PCA processed particles when prepared under nearly identical conditions of the other operating variables. More specifically, the planar precursors, Ni(II)(salen), give rise to primary particles with rod-like structures with submicron length scales and diameters of less than 100 nanometers. Deviations from planarity of the precursors produce substantial changes in particle structure, as illustrated by the 50 nm spherical particles prepared with Ru(salen)(NO)(Cl).

The ability to manipulate particle morphology by changing the molecular geometry of the starting material is also supported by previous work. A large majority of PCA processed particles have spherical-like morphologies, and these particles always consist of compounds with non-planar molecular structures. Conversely, rod-like structures are observed for the few cases when nearly planar organic compounds are used in processing. For instance, griseofulvin and carbamazepine, two organic pharmaceuticals having basically planar molecular structures, afford elongated micron-sized rods after PCA processing. See Reverchon E., Supercritical antisolvent precipitation of micro- and nano- particles, J. Supercrit. Fluids 15, 1-21 (griseofluvin) (1999); Edwards A. D., Yu Shekunov B., Kordikowski A., Forbes R. T., and York P., Crystallization of pure anhydrous polymorphs of carbamazepine by solution enhanced dispersion with supercritical fluids (SEDS), Journal of Pharmaceutical Sciences, 90, 1115-1124 (2001). Of course, neither reference observed that the planarity of the molecular structure affected particle morphology. Nor did either reference suggest changing the planarity of the molecular structure to affect particle morphology.

While particle formation using PCA technology is undoubtedly a complex process with several processing variables contributing to the final morphology of the particles, the present invention is directed to the surprising discovery that one controlling variable is the molecular structure of the precursor compounds. By making modifications to the core structure, particles whose morphology can be varied to enhance applications in absorption and catalysis may be produced.

From the foregoing it will be seen that this invention is one well adapted to attain all ends and objectives herein-above set forth, together with the other advantages which are obvious and which are inherent to the invention. Since many possible embodiments may be made of the invention without departing from the scope thereof, it is to be understood that all matters herein set forth or shown in the accompanying drawings are to be interpreted as illustrative, and not in a limiting sense. Further, while specific embodiments have been shown and discussed, various modifications may of course be made, and the invention is not limited to the specific forms or arrangement of parts and steps described herein, except insofar as such limitations are included in the following claims. Further, it will be understood that certain features and subcombinations are of utility and may be employed without reference to other features and subcombinations. This is contemplated by and is within the scope of the claims.

What is claimed and desired to be secured by Letters Patent is as follows:

1. A process for the production of nanoparticles, said process comprising the step of:
providing a first compound with a first molecular structure;
altering the planarity of the molecular structure of said first compound to form a second compound with a second molecular structure;
forming a solution including at least one solvent and at least one solute comprising said second compound with said second molecular structure;
spraying said solution containing said at least one solute through a nozzle into an antisolvent;
generating atomized droplets of said solution; and
contacting droplets with the antisolvent to form nanoparticles of said solute with a particle morphology.

2. The process of claim 1 wherein said first molecular structure is planar, and said second molecular structure is non-planar, and said nanoparticles have a spherical morphology.

3. The process of claim 1 wherein said solute is a metal-salen complex.

4. The process of claim 1 wherein said first compound having a first molecular structure comprises a nickel or cobalt salen having a planar structure, and said altering step comprises the addition of an axial group on a ring of said metal salen so that said second compound is non-planar.

5. The process of claim 1 wherein said first compound having a first molecular structure comprises a nickel or cobalt salen having a planar structure, and said altering step comprises the alteration of said ethylene linker of said metal salen so that said second compound is non-planar.

6. A process for the production of nanoparticulate metal complexes, said process comprising the step of:
providing a metal complex;
forming a solution including at least one solvent and said metal complex;
spraying said solution containing said at least one solvent and metal complex through a nozzle into an antisolvent;
generating atomized droplets of said solution; and
contacting droplets with the antisolvent to form a nanoparticulate metal complex.

7. The process of claim 6 wherein said metal complex is a nickel, cobalt, iron, or ruthenium salen.

8. The process of claim 6 wherein said metal complex is selected from the group consisting of a transition metal complexed with a salen, saltin, salophen, or salayhexin ligand.

9. A nanoparticulate metal complex having particle morphology comprising rods.

10. The nanoparticulate metal complex of claim 9 wherein said metal complex comprises a metal salen complex.

11. The nanoparticulate metal salen complex of claim 10 wherein said rods have an average diameter of about 85 nm and an average length of about 700 nm.

12. The nanoparticulate metal salen complex of claim 10 wherein said metal salen complex is either a nickel, cobalt, or ruthenium salen complex.

13. A nanoparticulate metal complex having a particle morphology comprising spheres.

14. The nanoparticulate metal complex claim 13 wherein said metal complex comprises a metal salen complex.

15. The nanoparticulate metal salen complex of claim 13 wherein the average particle diameter of said spheres is about 50 nm.

16. The nanoparticulate metal salen complex of claim 13 wherein said metal salen complex is either a nickel, cobalt, or ruthenium salen complex.

17. A process for altering the morphology of a nanoparticle comprising:
providing a first compound with a first molecular structure;
forming a nanoparticle of said first compound having a first particle morphology;
altering the planarity of said first compound to form a second compound having a second molecular structure; and
forming a nanoparticle having a second particle morphology, from said second compound having said second molecular structure, said second particle morphology being different than said first particle morphology.

18. The process of claim 17 wherein said first molecular structure is planar, and said second molecular structure is non-planar, said first particle morphology comprises rods, and said second particle morphology comprises spheres.
